(19) United States
(12) Patent Application Publication
(10) Pub. No.: US 2019/0010526 A1
(43) Pub. Date: Jan. 10, 2019

(54) METHODS OF PRODUCING EPOXIDIZED FATTY ACID ALKYL ESTERS USEFUL AS PLASTICIZERS

(71) Applicant: Arkema Inc., King of Prussia, PA (US)

(72) Inventors: Mohammad R. Kazemizadeh, Blooming Prairie, MN (US); David E. Maixner, Blooming Prairie, MN (US); Cecile N. Bonnet, Philadelphia, PA (US)

(21) Appl. No.: 16/066,317

(22) PCT Filed: Jan. 11, 2017
(86) PCT No.: PCT/US2017/012919

§ 371 Date: Jun. 27, 2018

Related U.S. Application Data
(60) Provisional application No. 62/278,075, filed on Jan. 13, 2016.

(51) Int. Cl. C12P 17/02; C07D 301/12; C08K 5/1515

(52) U.S. Cl. CPC C12P 17/02; C08K 5/1515; C07D 301/12

(57) ABSTRACT

Epoxidized fatty acid alkyl esters useful as plasticizers are obtained by reacting epoxidized fatty acid triglycerides with alcohol in the presence of an enzyme having transesterification activity.

### CROSS-REFERENCE TO RELATED APPLICATION

This application is related to and claims the benefit of U.S. Provisional Application No. 62/278,075 filed on Jan. 13, 2016, titled METHOD OF PRODUCING EPOXIDIZED FATTY ACID ALKYL ESTERS USEFUL AS PLASTICIZERS; the contents of which are incorporated herein by reference in their entirety for all purposes.

### FIELD OF THE INVENTION

The present invention relates to processes for producing epoxidized fatty acid alkyl esters, the epoxidized fatty acid alkyl esters thereby obtained and the use of such epoxidized fatty acid alkyl esters as plasticizers.

### BACKGROUND OF THE RELATED ART

In recent years, there has been growing interest in developing new products which are based on or derived from renewable resources, such as the triglyceride oils and fats obtainable from plant and animal sources. For example, epoxidized vegetable oils and epoxidized fatty acid alkyl esters have been widely used as plasticizers for polymers and, specifically, as replacements for phthalate plasticizers in flexible PVC formulations.

Epoxidized fatty acid alkyl esters, methods for making epoxidized fatty acid alkyl esters, and the use of epoxidized fatty acid alkyl esters as plasticizers are described, for example, in U.S. Pat. No. 3,070,608; U.S. Pat. No. 6,797,753; and US Patent Application Publications Nos. 2015/0337112 and 2015/0252014; processes for preparing non-epoxidized fatty acid alkyl esters are described, for example, in WO 2006/072256 and WO 2012/098114.

Current manufacturing processes for making epoxidized fatty acid alkyl esters are based on the direct epoxidation of unsaturated fatty alkyl esters using organic peracids such as performic or peracetic acid. Additionally, such epoxidized fatty acid alkyl esters can be produced through a transesterification of epoxidized vegetable oil using a base catalyst such as potassium hydroxide or sodium methoxide. However, such transesterification conditions typically result in the production of some ring-opened byproducts as a result of reaction of the epoxy groups present in the starting material; this is undesirable, since epoxy groups impart advantageous properties such as acid scavenging capability and other stabilizing effects. impart advantageous properties such as acid scavenging capability and other stabilizing effects.

Thus, the development of improved methods for synthesizing epoxidized fatty acid alkyl esters would be desirable.

### SUMMARY OF THE INVENTION

A first embodiment of the invention provides a process of making epoxidized fatty acid alkyl esters (which may also be referred to as “alkyl esters of epoxidized fatty acids”), wherein the process comprises reacting one or more epoxidized fatty acid triglycerides with one or more monohydric alcohols in the presence of at least one enzyme having transesterification activity.

In a second embodiment, the one or more monohydric alcohols is or are selected from the group consisting of C1-C10 alkanols.

In a third embodiment, the one or more monohydric alcohols is or are selected from the group consisting of methanol, ethanol, propanol, butanol, octanol and combinations thereof.

In a fourth embodiment, the at least one enzyme having transesterification activity includes at least one lipolytic enzyme.

In a fifth embodiment, the at least one enzyme having transesterification activity includes at least one enzyme selected from the group consisting of lipases, phospholipases and cutinases.

In a sixth embodiment, the epoxidized fatty acid triglycerides have been obtained by epoxidation of one or more unsaturated fatty acid triglycerides selected from vegetable oils and fats and animal oils and fats.

In a seventh embodiment, a reaction product is obtained which is comprised of epoxidized fatty acid alkyl esters and free fatty acids and the process comprises an additional step of converting at least a portion of the free fatty acids in the reaction product to fatty acid salts.

In an eighth embodiment, the fatty acid salts obtained by practice of the seventh embodiment are selected from the group consisting of alkaline earth and zinc salts of fatty acids.

In a ninth embodiment, the additional step of converting at least a portion of the free fatty acids to fatty acid salts comprises contacting the reaction product with at least one of calcium oxide or zinc oxide.

In a tenth embodiment, the reaction product after the additional step of converting at least a portion of the free fatty acids to fatty acid salts has an acid value less than 5 mg KOH/g.

In an eleventh embodiment, the epoxidized fatty acid alkyl esters have an oxirane oxygen content of from 2 to 12% by weight.

In a twelfth embodiment, the one or more epoxidized fatty acid triglycerides are obtained by epoxidizing one or more unsaturated fatty acid triglycerides with an organic peracid (which may be formed in situ using hydrogen peroxide and a carboxylic acid) or a combination of an organic acid and hydrogen peroxide.

In a thirteenth embodiment, the one or more epoxidized fatty acid triglycerides are obtained by epoxidizing one or more unsaturated fatty acid triglycerides with an organic peracid selected from the group consisting of peracetic acid and performic acid or a combination of an organic acid selected from the group consisting of acetic acid and formic acid and hydrogen peroxide.

In a fourteenth embodiment, the epoxidation is catalyzed by at least one catalyst selected from the group consisting of acids, metal catalysts and enzymes.

In a fifteenth embodiment, at least 1 mole monohydric alcohol per mole of fatty acid in the epoxidized fatty acid triglyceride is used.

In a sixteenth embodiment, at least 1.2 moles monohydric alcohol per mole of fatty acid in the epoxidized fatty acid triglyceride is used.

In a seventeenth embodiment, the one or more monohydric alcohols are added over a period of time to a mixture of the epoxidized fatty acid triglyceride(s) and enzyme(s).

In an eighteenth embodiment, the period of time over which the monohydric alcohol(s) is or are added is 8 hours or longer.

In a nineteenth embodiment, less than 10 weight % water, based on the total weight of epoxidized fatty acid triglyceride(s) and alcohol, is present during the reacting.

In a twentieth embodiment, the epoxidized fatty acid triglyceride(s) is or are selected from the group consisting of epoxidized vegetable oils and fats, epoxidized animal oils and fats and combinations thereof.

In a twenty-first embodiment, the epoxidized fatty acid triglyceride(s) is or are selected from the group consisting of epoxidized algae oil, epoxidized canola oil, epoxidized coconut oil, epoxidized castor oil, epoxidized corn oil, epoxidized cottonseed oil, epoxidized flax oil, epoxidized fish oil, epoxidized grapeseed oil, epoxidized hemp oil, epoxidized jatropha oil, epoxidized jojoba oil, epoxidized mustard oil, epoxidized canola oil, epoxidized palm oil, epoxidized palm stearin, epoxidized rapeseed oil, epoxidized safflower oil, epoxidized soybean oil, epoxidized sunflower oil, epoxidized tall oil, epoxidized olive oil, epoxidized tallow, epoxidized lard, epoxidized chicken fat, epoxidized linseed oil, epoxidized tung oil, epoxidized linseed oil, epoxidized tung oil and combinations thereof.

In a twenty-second embodiment, the process is carried out in a batch mode, a semi-continuous mode or a continuous mode.

In a twenty-third embodiment, the reacting is carried out under conditions effective to provide a reaction product comprised of 1% to 99% by weight epoxidized fatty acid triglycerides, based on the total weight of epoxidized fatty acid triglycerides, epoxidized fatty acid monoglycerides, epoxidized fatty acid diglycerides and epoxidized fatty acid alkyl esters present in the reaction product.

In a twenty-fourth embodiment, the reacting is carried out under conditions effective to provide a reaction product comprised of 30% to 70% by weight epoxidized fatty acid triglycerides, based on the total weight of epoxidized fatty acid triglycerides, epoxidized fatty acid monoglycerides, epoxidized fatty acid diglycerides and epoxidized fatty acid alkyl esters present in the reaction product.

In a twenty-fifth embodiment, the reacting is carried out in a two phase system wherein a first phase is comprised of the alcohol(s), enzyme, water and glycerol generated during transesterification and a second phase is comprised of the epoxidized fatty acid triglycerides and/or the epoxidized fatty acid alkyl esters.

In a twenty-sixth embodiment, the first phase and the second phase are mixed during the reacting using high shear mixing.

In a twenty-seventh embodiment, the process is conducted in a countercurrent mode.

In a twenty-eighth embodiment, the first phase is separated from the second phase following the reacting and then re-used, in whole or in part, in a further transesterification reaction.

In a twenty-ninth embodiment, glycerol is recovered from the first phase following the reacting.

In a thirtieth embodiment, the second phase is separated from the first phase following the reacting and treated with an alkaline agent to reduce the level of free fatty acids present in the second phase.

In a thirty-first embodiment, the second phase is separated from the first phase following the reacting and further treated with an immobilized lipase and alcohol to increase the content of epoxidized fatty acid alkyl esters.

In a thirty-second embodiment, the present invention provides a process of making epoxidized fatty acid alkyl esters, wherein the process comprises reacting a mixture of one or more unsaturated fatty acid triglycerides, one or more alcohols, and one or more active oxygen sources in the presence of at least one enzyme having transesterification activity and at least one enzyme having epoxidation activity or at least one enzyme having both transesterification activity and epoxidation activity.

In a thirty-third embodiment, the active oxygen source(s) is or are selected from the group consisting of hydrogen peroxide and percarboxylic acids.

In a thirty-fourth embodiment, the reacting is carried out under conditions effective to provide a reaction product comprised of 1% to 99% by weight epoxidized fatty acid triglycerides, based on the total weight of epoxidized fatty acid triglycerides, epoxidized fatty acid monoglycerides, epoxidized fatty acid diglycerides and epoxidized fatty acid alkyl esters present in the reaction product.

In a thirty-fifth embodiment, the reacting is carried out under conditions effective to provide a reaction product comprised of 30% to 70% by weight epoxidized fatty acid triglycerides, based on the total weight of epoxidized fatty acid triglycerides, epoxidized fatty acid monoglycerides, epoxidized fatty acid diglycerides and epoxidized fatty acid alkyl esters present in the reaction product.

In a thirty-sixth embodiment, the invention provides a composition comprising one or more epoxidized fatty acid alkyl esters obtained by a process in accordance with any of the above-mentioned embodiments.

In a thirty-seventh embodiment, the invention provides a plasticized polymer formulation, comprising at least one polymeric resin and a composition comprising one or more epoxidized fatty acid alkyl esters obtained by a process in accordance with any of the above-mentioned embodiments.

In a thirty-eighth embodiment, the invention provides a method of plasticizing a polymeric resin, comprising combining the polymeric resin with a composition comprising one or more epoxidized fatty acid alkyl esters obtained by a process in accordance with any of the above-mentioned embodiments.

### DETAILED DESCRIPTION OF CERTAIN EMBODIMENTS OF THE INVENTION

In one aspect of the invention, epoxidized fatty acid alkyl esters are prepared by a process comprising reacting one or more epoxidized fatty acid triglycerides with one or more monohydric alcohols in the presence of at least one enzyme having transesterification activity.

The epoxidized fatty acid triglycerides may be epoxidized vegetable oils and fats, epoxidized animal oils and fats, and combinations thereof, which may be produced by subjecting vegetable- and animal-derived fats and oils containing unsaturation (carbon-carbon double bonds) to epoxidation conditions so as to convert at least a portion of the unsaturation sites to epoxy groups. Such methods are well-known in the art and include, for example, epoxidation using peroxygen compounds such as peracids and enzyme-catalyzed epoxidation. Suitable epoxidized fatty acid triglycerides are also available from commercial sources, such as Arkema Inc. (under the brand name “Vikoflex”). The unsaturated triglyceride which is subjected to epoxidation may be crude or refined; it may be processed or treated prior to epoxidation in any way known in the art such as, for example, bleaching, steam stripping, partial hydrogenation, interesterification, deodorization, degumming, de-acidification and the like.

The triglyceride employed as a starting material for epoxidation may be any glycerol triester in which the three hydroxyl groups of glycerol are esterified with fatty acid, with at least a portion of the fatty acid being unsaturated. Minor amounts of mono- and/or triglycerides and/or free fatty acids may additionally present, although in certain embodiments the starting material for epoxidation is at least 95%, at least 97%, at least 99% or even 100% by weight triglyceride. The fatty acids present (in esterified form) in the triglyceride may be any fatty acid or combination thereof, although to provide sites of unsaturation capable of being converted to epoxy (oxirane) groups at least some portion of the fatty acid(s) should be unsaturated fatty acids such as oleic acid, linoleic acid, linolenic acid, myristoleic acid, and arachidonic acid and the like and combinations thereof. The fatty acid may, for example, contain six to 26 carbon atoms, in particular ten to 24 carbon atoms and may contain 0, 1, 2, 3 or more carbon-carbon double bonds. An individual fatty acid triglyceride molecule may contain 1, 2, or 3 unsaturated fatty acid groups (bound in ester form to glycerol), which may be the same as or different from each other; 0, 1 or 2 saturated fatty acid groups may be present in the fatty acid triglyceride. An “unsaturated fatty acid triglyceride,” as used herein, refers to a triglyceride containing at least one unsaturated fatty acid group per molecule. Suitable sources of unsaturated triglycerides include, but are not limited to, triglycerides selected from the group consisting of algae oil, canola oil, coconut oil, castor oil, corn oil, cottonseed oil, flax oil, fish oil, grapeseed oil, hemp oil, jatropha oil, jojoba oil, mustard oil, canola oil, palm oil, palm stearin, rapeseed oil, safflower oil, soybean oil, sunflower oil, tall oil, olive oil, tallow, lard, chicken fat, linseed oil, tung oil and combinations thereof (it being understood that such oils and fats may contain some fraction of triglyceride molecules in which all three fatty acids bound to glycerol are saturated fatty acids). The fatty acid triglyceride or mixture of fatty acid triglycerides may, for example, have an iodine value of at least 40, at least 50, at least 60, at least 70, at least 80, at least 90, at least 100, or at least 110 g I₂/100 g or even higher. The iodine value of the fatty acid triglyceride(s) used for epoxidation may be from 80 to 220 g I₂/100 g, for example.

The fatty acid triglyceride(s) may, in one embodiment, be epoxidized via contact with an acid and an aqueous peroxide solution to thereby produce an epoxidized reaction mixture comprising epoxidized fatty acid alkyl esters, residual acid, residual peroxide, and water. Suitable peroxides for use in epoxidizing the natural oil include aqueous solutions of hydrogen peroxide, peroxycarboxylic acids (peracids), and organic hydroperoxides. In one embodiment, the peroxide employed is a peracid, which may be formed in situ by the use of a carboxylic acid in combination with hydrogen peroxide.

Suitable acids for use in epoxidizing the fatty acid triglycerides include carboxylic acids, such as formic acid and acetic acid (used in combination with hydrogen peroxide); and peroxycarboxylic acids, such as performic acid and peracetic acid. Catalysts such as mineral acids, heterogeneous acid resins (including, for example, ion exchange resins such as Amberlite® IR 120, marketed by Dow Chemical Company) and metal catalysts (e.g., catalysts containing metals such as titanium, molybdenum and tungsten) may optionally be employed in the epoxidation. The catalyst may be homogeneous (soluble in the reaction medium) or heterogeneous (insoluble in the reaction medium, e.g., supported or immobilized).

An enzyme may be used to catalyze the desired epoxidation reaction of the unsaturated fatty acid triglyceride. For example, an unsaturated fatty acid triglyceride may be epoxidized using hydrogen peroxide or other peroxy species as an oxygen donor and, optionally, a carboxylic acid as an active oxygen carrier in the presence of an enzyme having epoxidation activity, such as a lipase.

Methods of epoxidizing unsaturated fatty acid triglycerides are known in the art and any of such methods may be adapted for use in connection with the present invention. Such methods are described, for example, in Saurabh et al., Epoxidation of Vegetable Oils: A Review, International Journal of Advanced Engineering Technology, E-ISSN 0976-3945 (available from technicaljournalsonline.com) and Milchert et al., Technological Aspects of Chemoenzymatic Epoxidation of Fatty Acids, Fatty Acid Esters and Vegetable Oils: A Review, Molecules, 2015, 20, 21481-21493, the disclosures of each of which are incorporated herein by reference in their entirety for all purposes.

In one or more embodiments, the epoxidation reaction is controlled so as to produce epoxidized fatty acid triglyceride having an iodine value in the range of up to 20, up to 15, up to 12, up to 10, up to 7, up to 5, up to 2 or 0 grams of iodine per 100 grams of epoxidized fatty acid triglyceride (“g I₂/100 g”). Iodine value is determined according to the American Oil Chemists' Society (“AOCS”) recommended practice Cd 1-25. Additionally, the controlled epoxidation reaction conditions can be selected so as to produce epoxidized fatty acid triglyceride having an oxirane oxygen content of at least 1 wt %, at least 2 wt %, at least 3 wt %, at least 4 wt %, at least 5 wt %, at least 6 wt %, at least 6.5 wt % or even higher, based on the entire weight of the epoxidized fatty acid triglyceride. In various embodiments, the epoxidized fatty acid triglyceride can have an oxirane oxygen content up to 15%, up to 12%, up to 10%, or up to 8% by weight, based on the entire weight of the epoxidized fatty acid triglyceride. For example, the epoxidized fatty acid triglyceride may have an oxirane oxygen content of 4 to 10% by weight. Oxirane oxygen content is determined according to AOCS recommended practice Cd 9-57.

In various embodiments of the invention, at least 70%, at least 75%, at least 80%, at least 85%, at least 90%, at least 95% or even 100% of the carbon-carbon double bonds originally present in the unsaturated fatty acid triglyceride starting material are converted to epoxy (oxirane) groups.

Following epoxidation, the residual acid, peroxide, and water may be removed from the epoxidized reaction mixture via layer separation and neutralization. Layer separation involves separation of an aqueous layer, which contains water, acids, peroxide, and possible traces of oil and esters, from an organic layer containing the epoxidized fatty acid triglyceride. To accomplish layer separation, the reaction mixture is allowed to settle and separate into two layers by density difference, and the bottom aqueous layer is disposed of while the top organic layer is processed further to obtain the desired product. Following layer separation, the residual acid can be neutralized, such as by contact with a sodium/bicarbonate solution. Thereafter, the organic layer can be washed one or more times with water. In an embodiment, the organic layer is washed repeatedly until it is neutral (having a pH of about 7). Thereafter, the washed mixture can be subjected to layer separation again, followed by application of vacuum to the top organic layer to remove residual water.

The transesterification conditions used in the process according to the present invention typically and advantageously are selected so as to result in little or no change in the oxirane oxygen content and iodine value of the epoxidized fatty acid triglyceride starting material. The present invention is capable of achieving a high degree of transesterification of the epoxidized fatty acid triglyceride while avoiding significant ring-opening of the epoxy groups due to hydrolysis and/or reaction with the alcohol. In various embodiments of the invention, an epoxidized fatty acid triglyceride is utilized having a relatively low iodine value, i.e., containing comparatively low amounts of unsaturation (carbon-carbon double bonds). For example, in various embodiments, the iodine value of the epoxidized fatty acid triglyceride is less than 15, less than 10, less than 7, less than 5 or even less than 2 g I₂/100 g. In other embodiments, the oxirane oxygen (epoxy) content of the epoxidized fatty acid triglyceride is at least 1%, at least 2%, at least 3%, at least 4%, at least 5% or at least 6% by weight. The epoxidized fatty acid triglyceride used as a starting material in certain aspects of the present invention may have an oxirane oxygen content of not more than 15%, not more than 12%, not more than 10% or not more than 8% by weight. For example, the oxirane oxygen content may be 1% to 15%, 2% to 12%, or 3% to 10% by weight.

In various embodiments of the invention, the epoxidized fatty acid triglyceride(s) is or are selected from the group consisting of epoxidized algae oil, epoxidized canola oil, epoxidized coconut oil, epoxidized castor oil, epoxidized corn oil, epoxidized cottonseed oil, epoxidized flax oil, epoxidized fish oil, epoxidized grapeseed oil, epoxidized hemp oil, epoxidized jatropha oil, epoxidized jojoba oil, epoxidized mustard oil, epoxidized canola oil, epoxidized palm oil, epoxidized palm stearin, epoxidized rapeseed oil, epoxidized safflower oil, epoxidized soybean oil, epoxidized sunflower oil, epoxidized tall oil, epoxidized olive oil, epoxidized tallow, epoxidized lard, epoxidized chicken fat, and combinations thereof.

Prior to use in the transesterification reaction, the epoxidized fatty acid triglyceride may be subjected to one or more pretreatment steps such as, for example, mineral acid neutralization (i.e., neutralization of any mineral acid present in the epoxidized fatty acid triglyceride), filtration and/or drying.

The alcohol(s) reacted with the epoxidized triglyceride(s) may be any monohydric organic compound or combination of monohydric organic compounds, in particular monohydric aliphatic alcohols. The alcohol(s) employed for transesterification is or are selected based on the desired alkyl substituent of the epoxidized fatty acid alkyl ester. Alcohols suitable for use in transesterification include, but are not limited to, C₁ to C₈ monohydric linear alcohols, such as methanol, ethanol, propanol, and butanol, or C₃ to C₈ branched alcohols, such as isopropanol, isobutanol, and 2-ethylhexanol, as well as combinations of two or more of these. In one embodiment, the alcohol is methanol, such that the resultant epoxidized fatty acid alkyl esters are epoxidized fatty acid methyl esters.

The molar ratio of alcohol to fatty acid (including the epoxidized fatty acid triglyceride-bound fatty acids) is typically, in various embodiments of the invention, at least 1:1 or at least 1.2:1 and/or not more than 4:1 and/or not more than 3:1 or not more than 2:1.

The transesterification reaction of the present invention may be catalyzed by one or more enzymes having transesterification activity, such as, for example, lipolytic enzymes. The one or more lipolytic enzymes may be selected, for example, from lipases, phospholipases, cutinases, acyltransferases or a mixture of one and more of lipase, phospholipase, cutinase and acyltransferase enzymes. The one or more lipolytic enzymes may be selected from the enzymes in enzyme classes EC 3.1.1, EC 3.1.4, and EC 2.3. The one or more lipolytic enzymes may also be a mixture of one or more lipases. The one or more lipolytic enzyme may include a lipase and a phospholipase. The one or more lipolytic enzymes may include a lipase of enzyme class EC 3.1.1.3. The one or more lipolytic enzymes may include a lipase known to have activity on triglycerides, such as those found in oils and fats derived from natural sources such as plants and animals.

A suitable lipolytic enzyme may be a polypeptide having lipase activity, e.g., one selected from the Candida antarctica lipase A (CALA) as disclosed in WO 88/02775, the C. antarctica lipase B (CALB) as disclosed in WO 88/02775 and shown in SEQ ID NO:1 of WO2008065060, the Thermomyces lanuginosus (previously Humicola lanuginosus) lipase disclosed in EP 258 068), the Thermomyces lanuginosus variants disclosed in WO 2000/60063 or WO 1995/22615, in particular the lipase shown in positions 1-269 of SEQ ID NO: 2 of WO 95/22615, the Hyphozyma sp. lipase (WO 98/018912), and the Rhizomucor miehei lipase (SEQ ID NO:5 in WO 2004/099400), a lipase from P. alcaligenes or P. pseudoalcaligenes (EP 218 272), P. cepacia (EP 331 376), P. glumae, P. stutzeri (GB 1,372,034), P. fluorescens, Pseudomonas sp. strain SD 705 (WO 95/06720 and WO 96/27002), P. wisconsinensis (WO 96/12012); a Bacillus lipase, e.g., from B. subtilis (Dartois et al. (1993), Biochemica et Biophysica Acta, 1131, 253-360), B. stearothermophilus (JP 64/744992) or B. pumilus (WO 91/16422). Also suitable are lipases from any of the following organisms: Fusarium oxysporum, Absidia reflexa, Absidia corymbefera, Rhizomucor miehei, Rhizopus delemar (oryzae), Aspergillus niger, Aspergillus tubingensis, Fusarium heterosporum, Aspergillus oryzae, Penicilium camembertii, Aspergillus foetidus, Aspergillus niger, Aspergillus oryzae and Thermomyces lanuginosus, such as a lipase selected from any of SEQ ID NOs: 1 to 15 in WO 2004/099400.

In one embodiment, a lipase used in the present invention is a lipase having a sequence identity to the mature polypeptide of SEQ ID NO: 2 of at least a polypeptide having at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or even 100% sequence identity to the polypeptide shown in positions 1-269 of SEQ ID NO: 2 of WO 95/22615 or to the polypeptide shown in SEQ ID NO:1 of WO2008/065060. Commercial lipase preparations suitable for use in the process of the invention include LIPOZYME CALB L, LIPOZYME® TL 100L and CALLERA™ TRANS (all available from Novozymes A/S). Especially suitable for use is the liquid enzyme product available commercially from Novozymes under the brand name Eversa® Transform.

The one or more lipolytic enzymes may include a polypeptide having phospholipase activity, such as phospholipase A₁, phospholipase A₂, phospholipase B, phospholipase C, phospholipase D, lyso-phospholipases activity, and/or any combination thereof. In the process of the present invention, the one or more lipolytic enzyme may be a phospholipase, e.g., a single phospholipase such as A₁, A₂, B, C, or D; two or more phospholipases, e.g., two phospholipases, including, without limitation, both type A and B; both type A₁ and A₂; both type A₁ and B; both type A₂ and B; both type A₁ and C; both type A₂ and C; or two or more different phospholipases of the same type.

The one or more lipolytic enzyme may be a polypeptide having phospholipase activity, as well as having acyltransferase activity, e.g., a polypeptide selected from the polypeptides disclosed in WO 2003/100044, WO 2004/064537, WO 2005/066347, WO 2008/019069, WO 2009/002480, and WO 2009/081094. Acyltransferase activity may be e.g., determined by the assays described in WO 2004/064537.

The phospholipase may be selected from the polypeptides disclosed in WO 2008/036863 and WO 20003/2758. Suitable phospholipase preparations include PURIFINE® (available from Verenium) and LECITASE® ULTRA (available from Novozymes A/S). An enzyme having acyltransferase activity is available as the commercial enzyme preparation LYSOMAX® OIL (available from Danisco A/S).

The one or more lipolytic enzyme may include, for example, a polypeptide having cutinase activity. The cutinase may, e.g., be selected from the polypeptides disclosed in WO 2001/92502, in particular the Humicola insolens cutinase variants disclosed in Example 2.

In certain embodiments of the invention, the one or more lipolytic enzymes include an enzyme having at least 60%, at least 70%, at least 80%, at least 90%, at least 95%, at least 96%, at least 97%, at least 98%, or even at least 99% identity to any of the aforementioned lipases, phospholipases, cutinases, and acyltransferases.

In additional embodiments, the one or more lipolytic enzyme has or have at least 60%, at least 70%, at least 80%, at least 90%, at least 95%, at least 96%, at least 97%, at least 98%, at least or even at least 99% identity to the amino acid sequence shown as positions 1-269 of SEQ ID NO: 2 of WO 95/22615.

The one or more lipolytic enzymes used in the process of the present invention may be derived or obtainable from any of the sources mentioned herein. The term “derived” means in this context that the enzyme may have been isolated from an organism where it is present natively, i.e., the identity of the amino acid sequence of the enzyme is identical to a native enzyme. The term “derived” also means that the enzymes may have been produced recombinantly in a host organism, the recombinant produced enzyme having either an identity identical to a native enzyme or having a modified amino acid sequence, e.g., having one or more amino acids which are deleted, inserted and/or substituted, i.e. a recombinantly produced enzyme which is a mutant and/or a fragment of a native amino acid sequence. Within the meaning of a native enzyme are included natural variants. Furthermore, the term “derived” includes enzymes produced synthetically by, e.g., peptide synthesis. The term “derived” also encompasses enzymes which have been modified, e.g., by glycosylation, phosphorylation etc., whether in vivo or in vitro. The term “obtainable” in this context means that the enzyme has an amino acid sequence identical to a native enzyme. The term encompasses an enzyme that has been isolated from an organism where it is present natively, or one in which it has been expressed recombinantly in the same type of organism or another, or enzymes produced synthetically by e.g., peptide synthesis. With respect to recombinantly produced enzyme the terms “obtainable” and “derived” refers to the identity of the enzyme and not the identity of the host organism in which it is produced recombinantly.

Accordingly, the one or more lipolytic enzymes may be obtained from a microorganism by use of any suitable technique. For instance, an enzyme preparation may be obtained by fermentation of a suitable microorganism and subsequent isolation of an enzyme preparation from the resulting fermented broth or microorganism by methods known in the art. The enzyme may also be obtained by use of recombinant DNA techniques. Such method normally comprises cultivation of a host cell transformed with a recombinant DNA vector comprising a DNA sequence encoding the enzyme in question and the DNA sequence being operationally linked with an appropriate expression signal such that it is capable of expressing the enzyme in a culture medium under conditions permitting the expression of the enzyme and recovering the enzyme from the culture. The DNA sequence may also be incorporated into the genome of the host cell. The DNA sequence may be of genomic, cDNA or synthetic origin or any combinations of these, and may be isolated or synthesized in accordance with methods known in the art.

The one or more lipolytic enzymes may be applied or utilized in any suitable formulation or form, e.g., as lyophilized powder or in aqueous solution. Immobilized enzymes may also be employed.

The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows:

(Identical Residues.times.100)/(Length of Alignment-Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the—nobrief option) is used as the percent identity and is calculated as follows:

(Identical Deoxyribonucleotides.times.100)/(Length of Alignment-Total Number of Gaps in Alignment)

The amount of enzyme present during the transesterification reaction is not believed to be critical and will depend, among other factors, on the particular enzyme(s) selected for use. Typically, however, the amount of enzyme may be, in certain embodiments of the invention, from 0.05 to 1% by weight based on the weight of the epoxidized fatty acid triglyceride substrate.

The transesterification, in one embodiment of the invention, may be carried out using a procedure comprising forming a two phase reactant mixture comprising epoxidized fatty acid triglyceride (comprising an “oil” phase) and alcohol and water (comprising an “aqueous” phase) and contacting the reactant mixture with one or more enzymes capable of catalyzing transesterification (in one embodiment, the enzyme is present in the aqueous phase). A portion of the alcohol may also be present in the oil phase, admixed with the epoxidized fatty acid triglycerides. The interactions between the epoxidized fatty acid triglyceride, alcohol and enzyme generally take place at the interphase layer between the oil and aqueous phases. The two phases may be subjected to periodic or continuous agitation, wherein the two phases are intimately mixed. The two phases in the reactant mixture may, for example, be mixed using a high shear mixer or a cavitator or through the use of eductors. Such agitation helps to disperse the aqueous phase in the form of small droplets within a continuous oil phase, increasing the contact surface area between the oil phase and the aqueous phase; this typically will increase the rate of the desired transesterification reaction. Under certain conditions, a pseudo three-phase system may be created, wherein an emulsified interphase layer phase exists between the oil phase and the aqueous phase. The process may be carried out in a batch operation manner, as a continuous stirred tank reactor system, or in a counter-current mode (including a continuous, counter-current process). As the transesterification proceeds, glycerol is generated from the epoxidized fatty acid triglyceride and accumulates predominantly in the aqueous phase and epoxidized fatty acid alkyl ester is generated from the reaction of alcohol and epoxidized fatty acid triglyceride and accumulates predominantly in the oil phase.

Generally speaking, it will be advantageous to limit the amount of water present in the above-mentioned two phase system, although a sufficient amount is typically used to form an aqueous phase in addition to the phase containing the epoxidized fatty acid triglycerides. For example, less than 25 weight %, less than 20 weight %, less than 15 weight %, 10 weight %, or less than 5 weight % water based on the total weight of epoxidized fatty acid triglyceride(s) and alcohol(s), is employed in various embodiments of the invention. In other embodiments, an amount of water is utilized which is at least 0.5 weight % or at least 1 weight % based on the total weight of epoxidized fatty acid triglyceride(s) and alcohol(s).

All of the components of the reactant mixture may be combined together prior to transesterification being commenced. In a desirable embodiment, however, the alcohol(s) are added to the other components, either stepwise or continuously. The addition of alcohol may take place over an extended period of time, e.g., over at least 1, 2, 3, 4, 5, 6, 7 or 8 hours but not more than 24, 20, 18, 15 or 12 hours.

Typically, the reactant mixture during transesterification is maintained at approximately room temperature or temperatures somewhat above room temperature. For example, the transesterification temperature may be within the range of from 15° C. to 60° C. The transesterification temperature should be selected so as to avoid deactivation of the enzyme having transesterification activity while maintaining a commercially acceptable rate of transesterification.

The reaction of epoxidized fatty acid triglyceride and alcohol, catalyzed by enzyme, is carried out for a period of time effective to achieve the desired degree of conversion of the epoxidized fatty acid triglyceride to the epoxidized fatty acid alkyl ester. Typically, this period of time is from 5 hours to 40 hours (including the time during which the alcohol is being added to the epoxidized fatty acid triglyceride, if such an addition is practiced). In the embodiment where the alcohol is added over a period of time, it will generally be desirable to continue the transesterification reaction for at least a few hours after alcohol addition is completed, e.g., 5 to 30 hours.

In one embodiment of the invention, the transesterification reaction is carried out in a plurality of stages wherein in a first stage the epoxidized fatty acid triglyceride, alcohol and a first portion of enzyme are reacted for a period of time effective to achieve partial transesterification of the epoxidized fatty acid triglyceride. The partially transesterified reaction product is then separated and subjected, in a second stage, to further transesterification catalyzed by a second portion of enzyme having transesterification activity. If the separated partially transesterified reaction product is deficient in alcohol, additional alcohol may be introduced in the second stage to achieve the desired molar ratio of alcohol to glyceride-bound fatty acid (e.g., at least 1:1 or at least 1.2:1). The second portion of enzyme may be any of the types of lipolytic enzymes previously described and may be the same as or different from the enzyme used in the first transesterification stage. In one embodiment, the second portion of enzyme is an immobilized lipase. The second stage is continued for a time and at a temperature effective to achieve the desired degree of transesterification.

In various embodiments of the invention, reaction of the epoxidized fatty acid triglyceride and alcohol, catalyzed by enzyme, is continued (either in one stage or in multiple stages) until the yield of epoxidized fatty acid alkyl esters is at least 60%, at least 65%, at least 70%, at least 75%, at least 80%, at least 85%, at least 90%, at least 95% or even higher.

Without wishing to be bound by theory, the transesterification reaction is believed to typically proceed in a manner such that each of the three fatty acid ester groups on an individual molecule of epoxidized fatty acid triglyceride is successively cleaved so as to generate a hydroxyl group attached to a glycerol moiety and an epoxidized fatty acid alkyl ester resulting from the reaction of a monohydric alcohol with a fatty acid moiety originally bonded to a glycerol moiety in the form of an ester group. Accordingly, epoxidized fatty acid monoglycerides and epoxidized fatty acid diglycerides may be generated as transesterification reaction intermediates (which may also be present in the final reaction product, depending upon the extent to which the starting epoxidized fatty acid triglyceride is transesterified). In addition, free fatty acids (including epoxidized free fatty acids) may be formed as a result of enzyme-catalyzed hydrolysis of the tri-, di- and/or monoglycerides. It is also possible that unreacted epoxidized fatty acid triglyceride may remain in the reaction product obtained by practice of the transesterification process of the present invention. Generally speaking, the proportion of epoxidized fatty acid alkyl ester present in the reaction product relative to the total amount of unreacted epoxidized fatty acid triglyceride+epoxidized fatty acid monoglyceride+epoxidized fatty acid diglyceride will increase as the reaction time is increased. It may be advantageous to terminate the transesterification reaction at a point in time wherein some of the starting epoxidized fatty acid triglyceride remains unreacted (e.g., at least 1% but no more than 99% or at least 30% but no more than 70%), as in some plasticized polymeric resin formulations the use as a plasticizer of a composition containing epoxidized fatty acid triglyceride in combination with epoxidized fatty acid alkyl ester may be preferred or advantageous.

In other embodiments, the transesterification reaction is permitted to proceed for a time effective to provide a reaction product wherein the amount of epoxidized fatty acid alkyl ester is at least 1%, at least 10%, at least 20%, at least 40%, at least 50%, at least 60%, at least 70%, at least 80% or at least 90%, based on the total weight of unreacted epoxidized fatty acid triglyceride+epoxidized fatty acid monoglyceride+epoxidized fatty acid diglyceride+epoxidized fatty acid alkyl ester.

The reaction product containing the desired epoxidized fatty acid alkyl ester(s) may potentially be refined or purified by methods known in the art such as distillation (including flash evaporation, stripping and deodorization); phase separation; filtration; extraction; and/or drying. For example, the product may be dried to remove residual alcohol and/or water; phase separated to remove residual alcohol, glycerol co-product and water; and/or distilled to remove water, glycerol co-product, residual alcohol and/or other components more volatile than the epoxidized fatty acid alkyl esters. In one embodiment, the glycerol co-product is recovered from the aqueous phase by removing water and residual alcohol by a suitable method such as distillation or flash stripping. The separated water and alcohol may be reused in a further transesterification reaction of epoxidized fatty acid triglyceride. The glycerol may be subjected to further purification, such as by treatment with ion exchange or adsorption resins and/or activated carbon. The layer containing the epoxidized fatty acid alkyl ester(s) may be subjected to drying, evaporation or distillation so as to remove more volatile components such as water and alcohol.

In one embodiment, the enzyme remains with the water, glycerol and unreacted alcohol in the aqueous phase and the recovered aqueous phase containing enzyme is reused in a subsequent transesterification reaction. One or more components of the aqueous phase may be separated, in whole or in part, from the aqueous phase prior to recycling and reuse of the enzyme.

If the reaction product contains free fatty acid (which will accumulate predominantly in the oil phase), it generally will be desirable to substantially or entirely remove the free fatty acid (by a suitable technique such as alkaline/caustic washing followed by water washing, or the like) or to convert the free fatty acid to the salt form, with the fatty acid salt(s) being left in the epoxidized fatty acid alkyl ester product. In one embodiment, the fatty acid salt(s) formed are salts which are capable of functioning as stabilizers for polymeric resins such as polyvinyl chloride resins and the like. For example, the salt may be an alkaline earth or zinc salt, in particular, a calcium, barium, magnesium or zinc salt or combination thereof.

Conversion of free fatty acid to the above-mentioned salt(s) may be carried out using any suitable method, such as by heating the reaction product with an oxide or hydroxide of zinc or an alkaline earth (e.g., zinc oxide, calcium oxide) for a time and at a temperature effective to react the carboxylic acid group of the free fatty acid. In one embodiment, a stoichiometric excess of the oxide or hydroxide is used. If the oxide or hydroxide is in solid particulate form, any portion left unreacted following conversion of the free fatty acid may be removed by filtration or other suitable separation technique. At least a portion of the fatty acid salt formed may be solubilized in the reaction product and thus not removed by such a filtration step. The dissolved fatty acid salts may function as stabilizers/acid scavengers when the epoxidized fatty acid alkyl esters are employed as plasticizers in PVC and other polymeric resin formulations.

According to one embodiment of the invention, epoxidized fatty acid alkyl esters are prepared by a process comprising reacting a mixture of one or more unsaturated fatty acid triglycerides, one or more alcohols, and one or more active oxygen sources (e.g., hydrogen peroxide) in the presence of at least one enzyme having transesterification activity and at least one enzyme having epoxidation activity or at least one enzyme having both transesterification activity and epoxidation activity. Epoxidation and transesterification thus take place within the same reactor, thereby simplifying the overall process by eliminating the need to conduct these chemical transformations in distinct stages or steps. Generally speaking, this embodiment of the invention may be carried out using the conditions and procedures described herein as being suitable for the transesterification of already-epoxidized fatty acid triglycerides with alcohol, with the exception that an effective amount of an active oxygen source (e.g., a peroxy compound, such as hydrogen peroxide) is additionally present.

In one embodiment of the invention, the epoxidized fatty acid alkyl ester has a relatively low iodine value, i.e., it contains comparatively low amounts of unsaturation (carbon-carbon double bonds). For example, in various embodiments, the iodine value of the epoxidized fatty acid alkyl ester product is less than 15, less than 10, less than 7, less than 5 or even less than 2 g I₂/100 g.

In other embodiments, the oxirane oxygen (epoxy) content of the epoxidized fatty acid alkyl ester product is at least 1%, at least 2%, at least 3%, at least 4%, at least 5% or at least 6% by weight. The epoxidized fatty acid alkyl esters in accordance with the present invention may, in additional embodiments, have oxirane oxygen contents of not more than 15%, not more than 12%, not more than 10% or not more than 8% by weight. For example, the oxirane oxygen content may be 1% to 15%, 2% to 12%, or 3% to 10% by weight. For certain end use applications, it may be desirable to provide epoxidized fatty acid alkyl esters having relatively low levels of free fatty acid. In various embodiments of the invention, the acid value may be, for instance, less than 15, less than 12, less than 10, less than 7, less than 5, less than 3, less than 1 or even 0 mg KOH/g.

The epoxidized fatty acid alkyl esters of the present invention have utility as plasticizers and stabilizers. A plasticizer is a substance that can lower the modulus and tensile strength, and increase flexibility, elongation, impact strength, and tear strength of a polymeric resin (typically a thermoplastic polymer) to which it is added. A plasticizer may also lower the melting point of the polymeric resin, which lowers the glass transition temperature and enhances processability of the polymeric resin to which it is added. In an embodiment, the present plasticizer is a phthalate-free plasticizer, or is otherwise void or substantially void of phthalate. The epoxidized fatty acid alkyl esters may function as stabilizers in polymeric resins and other compositions, as the epoxy functionality provides heat and light stability and is capable of scavenging acids.

One aspect of the present invention provides a polymeric composition which comprises at least one polymeric resin and one or more epoxidized fatty acid alkyl esters as described above. The epoxidized fatty acid alkyl esters function to plasticize and/or stabilize the polymeric resin(s).

Non-limiting examples of suitable polymeric resins include polysulfides, polyurethanes, acrylics, epichlorohydrins, nitrile rubbers, chlorosulfonated polyethylenes, chlorinated polyethylenes, polychloroprenes, styrene butadiene rubbers, natural rubbers, synthetic rubbers, EPDM rubbers, propylene-based polymers, ethylene-based polymers, and vinyl chloride resins. The term, “propylene-based polymer,” as used herein, is a polymer that comprises a majority weight percent polymerized propylene monomer (based on the total amount of polymerizable monomers), and optionally may comprise at least one polymerized comonomer. The term, “ethylene-based polymer,” as used herein, is a polymer that comprises a majority weight percent polymerized ethylene monomer (based on the total weight of polymerizable monomers), and optionally may comprise at least one polymerized comonomer.

The term “vinyl chloride resin,” as used herein, is a vinyl chloride polymer, such as polyvinyl chloride (“PVC”), or a vinyl chloride copolymer such as vinyl chloride/vinyl acetate copolymer, vinyl chloride/vinylidene chloride copolymer, vinyl chloride/ethylene copolymer or a copolymer prepared by grafting vinyl chloride onto ethylene/vinyl acetate copolymer. The vinyl chloride resin can also include a polymer blend of the above-mentioned vinyl chloride polymer or vinyl chloride copolymer with other miscible or compatible polymers including, but not limited to, chlorinated polyethylenes, thermoplastic polyurethanes, olefin polymers such as a methacryl polymer or acrylonitrile-butadiene-styrene polymer.

In one embodiment, the vinyl chloride resin is PVC.

Any suitable amount of epoxidized fatty acid alkyl ester in accordance with the present invention may be combined with a polymeric resin. For example, from 1 to 150 parts by weight epoxidized fatty acid alkyl ester per 100 parts by weight polymeric resin may be employed. In another embodiment, the polymeric composition is comprised of from 30 wt % to 70 wt % polymeric resin (e.g., PVC), from 5 wt % to 40 wt % epoxidized fatty acid alkyl ester in accordance with or produced in accordance with the present invention, and, optionally, up to 35 wt % of one or more additional additives such as filler.

The polymeric composition may include one or more of the following optional additives: a filler, a flame retardant, a heat stabilizer, an anti-drip agent, a colorant, a lubricant, a low molecular weight polyethylene, a hindered amine light stabilizer, a UV light absorber, a curing agent, a booster, a retardant, a processing aid, a coupling agent, an antistatic agent, a nucleating agent, a slip agent, a viscosity control agent, a tackifier, an anti-blocking agent, a surfactant, an extender oil, an acid scavenger, a metal deactivator, and any combination thereof.

In an embodiment, the polymeric composition includes PVC, epoxidized fatty acid alkyl ester in accordance with or made in accordance with the present invention as plasticizer, at least one filler (e.g., calcium carbonate, clays, silica, and any combination thereof), one or more metal soap stabilizers (e.g., zinc stearate or mixed metal stabilizers containing Ca, Zn, Mg, Sn, and any combination thereof), one or more phenolic or related antioxidants, and one or more processing aids.

Epoxidized fatty acid alkyl ester according to the present invention may be the sole type of plasticizer in the polymeric composition or may be present in combination with one or more other types of plasticizers such as, for example, epoxidized fatty acid alkyl esters other than those in accordance with the present invention, epoxidized triglycerides, phthalate plasticizers and the like and combinations thereof.

Epoxidized fatty acid alkyl esters in accordance with or obtained in accordance with the present invention are also useful as pigment dispersing agents, as acid/mercaptan scavenging agents, and as reactive diluents. Functional fluids, flavor and fragrance compositions, sealants, coatings and the like may be formulated using the epoxidized fatty acid alkyl esters.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

### EXAMPLES

### Example 1

In this example, a combination of formic acid and hydrogen peroxide was used to epoxidize soybean oil. The epoxidized soybean oil had the following analysis: oxirane oxygen=7.18% by weight, iodine value=0.71 g I₂/100 g and acid value=0.44 mg KOH/g. A 500 g portion of this oil was treated with dilute caustic to remove residual acid. It was then washed and dried under vacuum, thereby obtaining an oil having an acid value of 0.01 mg KOH/g. This oil was then mixed with 10 g of water and 1.5 g enzyme (Eversa® Transform enzyme solution, available from Novozymes) and while the resulting mixture was agitated 80 g of methanol was added slowly over 10 hours. The reaction was continued for 24 hours while maintaining the temperature of the mixture at 35° C. After this period, a GC analysis of the mixture indicated that 90% fatty acid methyl ester of the epoxidized soybean oil had been formed. The mixture was then transferred to a separatory funnel; the glycerol-containing layer at the bottom of the separatory funnel was separated and the upper layer (containing methyl esters of epoxidized soybean fatty acids) was washed with water and dried under vacuum. The product thereby obtained had the following characteristics: oxirane oxygen=6.83% by weight; iodine value=0.7 g I₂/100 g; acid value=16 mg KOH/g; and viscosity=62 cps at 25° C. After washing with 4% caustic solution, the acid value of the product (methyl esters of epoxidized soybean fatty acids) was reduced to 0.2 mg KOH/g.

### Example 2

In this experiment, 30% peracetic acid was used to epoxidize soybean oil. The epoxidized soybean oil that was used in an enzymatic transesterification reaction with methanol as the alcohol reactant had an oxirane oxygen content of 7.25% by weight, an iodine value of 0.5 g I₂/100 g, an acid value of 0.15 mg KOH/g and a viscosity of 389 cps at 25° C. The enzymatic conversion of this epoxidized soybean oil resulted in a 70% yield of epoxidized soy methyl ester.

The enzymatic transesterification reaction was conducted as follows. 500 g of the above-described epoxidized soybean oil was mixed with 10 g water and 1.5 g enzyme (Eversa® Transform liquid enzyme, supplied by Novozymes). Then, 80 g methanol was added over 10 hours while vigorously agitating the mixture. After 24 hours of mixing, the phases were separated. The upper (oil) phase was washed with water and then dried under vacuum. The soy methyl ester epoxide obtained had an oxirane oxygen content of 7.05% by weight, an iodine value of 0.5 g I₂/100 g, an acid value of 35 mg KOH/g and a viscosity of 38 cps at 25° C.

### Example 3

In this experiment, a commercial grade of epoxidized soybean oil that is available from Arkema Inc. under the trade name of Vikoflex® 7170 was used as the transesterification substrate. This epoxidized soybean oil had an oxirane oxygen content of 7.13% by weight, an iodine value of 1.15 g I₂/100 g, an acid value of 0.12 mg KOH/g, and a viscosity of 420 cps at 25° C. The soy methyl ester epoxide obtained following transesterification with methanol (using the same conditions as described in Example 1) had an oxirane oxygen content of 7.01% by weight, an iodine value of 1.7 g I₂/100 g, an acid value of 10.4 mg KOH/g, and a viscosity of 29 cps at 25° C.

### Example 4

In this experiment, an alternative neutralization procedure was used to reduce the acid number of the transesterification product containing methyl esters of epoxidized soybean fatty acids, which typically have relatively high acid numbers in the range of from 10 to 50 mg KOH/g. Reducing the acid number is expected to make the product more suitable for use as a plasticizer.

A 100 g portion of epoxidized soy methyl ester product obtained using an enzyme-catalyzed transesterification reaction with methanol having an acid value of 18 mg KOH/g was mixed with calcium oxide, steam stripped for 0.5 hour, then dried and filtered. The product obtained had an acid value of 0 mg KOH/g and a fatty acid soap content of 9450 ppm. The same procedure was repeated using ZnO and the same results were obtained.

### Example 5

Another procedure which may be used to reduce the acid value of the transesterification reaction product is to heat the product at a temperature above 150° C. under vacuum or at atmospheric pressure. Preferably, the product is heated at a temperature of 180° C. for a period of 1-4 hours under vacuum. In this example, 100 grams soy methyl ester epoxide having an acid value of 16 mg KOH/g, an oxirane oxygen content of 6.8% by weight and a viscosity of 35 cps at 25° C. was heated at 180° C. for 45 minutes. After this time, the acid value was found to have dropped to 5 mg KOH/g, viscosity increased to 55 cps at 25° C. and the oxirane oxygen content dropped to 5.5% by weight. IR indicated the hydroxyl peak related to the carboxylic group of fatty acid disappeared.

### Example 6

In this example, lime or caustic was used to reduce the acid value of soy methyl ester epoxide. A slurry of 1 gram calcium oxide with 3-5 grams of water was added to 100 gram soy methyl ester epoxide obtained from an Eversa® Transform-catalyzed transesterification process having an acid number of 16 mg KOH/g. The mixture was agitated for 5 minutes, dried under vacuum at 110° C. and then filtered to remove the excess lime. The filtrate was a product with an acid value of 0.55 mg KOH/g and a soap (fatty acid salt) content of 8568 ppm.

The same experiment was repeated, but instead of lime a 50 gram portion of 2% caustic solution was mixed with 100 g of the soy methyl ester epoxide, mixed for 5 minutes at 80° C. and then separated. The oil layer was washed and stripped at 110° C. under vacuum. The final product had an acid value of 0.25 mg KOH/g and a soap content of 98 ppm.

### Example 7

This example demonstrates the feasibility of carrying out epoxidation and transesterification in a “one pot” reaction.

### Example 7-1

Soybean oil (400 g) was charged to a 1 liter reaction vessel and heated to 35° C. Water (8 g) was added to the oil. The oil was then mixed vigorously (1480 rpm) for a minimum of 1 hr. Novozymes Eversa® Transform (1.2 g; for ester formation) and 4.0 g. Novozymes 435 (a Candida Antarctica lipase B immobilized on an acrylic resin hydrophobic carrier, for epoxidation) was then charged to the oil/water mixture. Vigorous agitation was maintained. Methanol (64 g) and 30% hydrogen peroxide (320 g) were charged to the mixture over a period of 10-12 hours. The reaction was then followed by iodine value analysis and G.C. analysis for methyl ester formation. When the iodine value was less than or equal to 3 g I₂/100 g, the reaction was settled.

The top oil layer was water washed at 35° C. three times (200 mls. each). Heptane (200 g) was added to aid separation. The oil was then stripped at 110° C. for 1 hour. Finally, the oil was filtered (Final Oil A).

The above procedure was repeated in Examples 7-2 and 7-3, with the variations noted in the table below.

| | Example 7-1 | Example 7-2 | Example 7-3 |
|---|---|---|---|
| Variation of above | none | 400 g.Heptane added | 400 ppm Calcium |
| procedure | | 50/50 to SBO | stearate added |
| Final epoxide mixture | Oxirane = 7.20 wt % | Oxirane = 2.40 wt % | Oxirane = 7.29 wt % |
| | I.V. = 3.5 g I₂/100 g | I.V. = 95.1 g I₂/100 g | I.V. = 4.5 g I₂/100 g |
| | A.V. = 37.0 mg | | Visc. = 77 cps @ 25° C. |
| | KOH/g | | A.V. = 37.0 mg KOH/g |
| | Peroxide Value = 0 | | Peroxide value = 0 |
| | Soy fatty acid methyl | | Color = 236 APHA |
| | ester epoxide content = | | Soy fatty acid methyl |
| | 43% | | ester epoxide content = |
| | Epoxidized soybean | | 45% |
| | oil content = 47% | | Epoxidized soybean oil |
| | Liquid at 40-50° C. | | content = 55% |
| | | | Liquid at 40-50° C. |

### Example 8

In this example, the performance of various epoxidized fatty acid-based compositions as plasticizers in polyvinylchloride formulations was evaluated.

The compositions tested were as follows:

### Sample 8-1 (Comparative)

This sample was prepared by epoxidizing vegetable oil using a combination of formic acid and hydrogen peroxide and then conducting a transesterification reaction with methanol catalyzed by sodium methoxide.

### Sample 8-2

This sample was prepared in accordance with the present invention, wherein an epoxidized vegetable oil was prepared using a combination of acetic acid, sulfuric acid and hydrogen peroxide and then subjecting the epoxidized vegetable oil to transesterification with methanol catalyzed by Eversa® Transform lipolytic enzyme.

### Sample 8-3

This sample was prepared in accordance with the procedure used for Sample 8-2, except that the transesterification reaction product obtained was heated to 180° C.

### Sample 8-4

This sample was prepared in accordance with the procedure used for Sample 8-2, except that the transesterification reaction product obtained was filtered prior to use as a plasticizer.

The attributes of Samples 8-1 to 8-4 are provided in the following table.

| | Oxirane | Iodine | | | |
|---|---|---|---|---|---|
| | Oxygen, | Value, g | Acid Value, | Color, | Viscosity, |
| Sample | wt % | I₂/100 g | mg KOH/g | APHA | cps @ 25° C. |
| 8-1 | 6.56 | 2.25 | 0.69 | 1 | 19.2 |
| 8-2 | 6.66 | 0.87 | 5.8 | 248 | 45.3 |
| 8-3 | 6.16 | 0.87 | 2.7 | 667 | 55.5 |
| 8-4 | 6.67 | 0.87 | 6.8 | 139 | 35.7 |

Each of Samples 8-1 to 8-4, as well as dioctyl phthalate (DOP) and diisononyl phthalate (DINP), was used as a plasticizer in the following plastisol formulation:

100 phr PVC homopolymer dispersion resin

40 phr Sample 8-1, 8-2, 8-3 or 8-4 or DOP or DINP

3 phr Epoxidized Soybean Oil

2 phr Barium Zinc heat stabilizer

The flexible PVC products made from the above formulation can be fabricated through a multi-step process. Samples of flexible PVC vinyl compounds were prepared as follows: 40 phr of Sample 8-1, 8-2, 8-3 or 8-4 was added in a Hobart mixer with a 5 quart capacity. 100 phr of the PVC resin was added slowly, mixing for few minutes, followed by the addition of 3 phr Epoxidized Soybean Oil and 2 phr Barium Zinc heat stabilizer into the mixture.

The plastisol formulations were converted to the plasticized PVC sheets using a hot press (190° C., 10 min, 10000 lbs). Fused test samples 80 mils thick are typically produced for most of the testing. The following properties are generally measured on the plasticized PVC to evaluate the useful of the material: hardness, modulus of flexibility, low temperature flexibility and volatility.

The properties of the plasticized PVC sheets obtained are shown in the following table:

| | | Tensile | | 100% | Brittleness |
|---|---|---|---|---|---|
| | Hardness, | Strength, | | Modulus, | Temp., |
| Sample | Shore A | psi | Elongation, % | psi | ° C. |
| 8-1 | 74 | 3047 | 375 | 1178 | −38 |
| 8-2 | 79 | 3100 | 347 | 1551 | −33 |
| 8-3 | 80 | 3214 | 350 | 1553 | −33 |
| 8-4 | 78 | 3063 | 341 | 1475 | −33 |
| DOP | 79 | 3011 | 291 | 1852 | −27 |
| DINP | 85 | 3141 | 287 | 2227 | −28 |

Based on the results above, it is noticeable that the plasticized PVC sheets prepared using Samples 8-2, 8-3 and 8-4 show similar, if not better (hardness shore A and brittleness temperature), performance as compared to commercial plasticizers such as DOP and DINP.

The acid value of the epoxidized fatty acid alkyl ester product obtained using the enzyme-catalyzed transesterification process is higher than the product obtained by sodium methoxide-catalyzed transesterification, but this not not seem to have much effect on the performance of the plasticized PVC sheets since the results are relatively similar between the product obtained from a sodium methoxide-catalyzed process (sample 8-1) and the products obtained from an enzyme-catalyzed process (Samples 8-2, 8-3 and 8-4). This example demonstrates the feasibility of using an enzyme-catalyzed transesterification process for making epoxidized fatty acid esters suitable for use as plasticizers for polymeric resins.

1. A process of making epoxidized fatty acid alkyl esters, wherein the process comprises reacting one or more epoxidized fatty acid triglycerides with one or more monohydric alcohols in the presence of at least one enzyme having transesterification activity.

2. The process of claim 1, wherein the one or more monohydric alcohols is or are selected from the group consisting of C1-C10 alkanols.

3. The process of claim 1, wherein the one or more monohydric alcohols is or are selected from the group consisting of methanol, ethanol, propanol, butanol, octanol and combinations thereof.

4. The process of any of claim 1 wherein the at least one enzyme having transesterification activity includes at least one lipolytic enzyme.

5. The process of claim 1 wherein the at least one enzyme having transesterification activity includes at least one enzyme selected from the group consisting of lipases, phospholipases and cutinases.

6. The process of claim 1 wherein the epoxidized fatty acid triglycerides have been obtained by epoxidation of one or more unsaturated fatty acid triglycerides selected from vegetable oils and fats and animal oils and fats.

7. The process of claim 1 wherein a reaction product is obtained which is comprised of epoxidized fatty acid alkyl esters and free fatty acids and the process comprises an additional step of converting at least a portion of the free fatty acids in the reaction product to fatty acid salts.

8. The process of claim 7, wherein the fatty acid salts are selected from the group consisting of alkaline earth and zinc salts of fatty acids.

9. The process of claim 7 wherein the additional step of converting at least a portion of the free fatty acids to fatty acid salts comprises contacting the reaction product with at least one of calcium oxide or zinc oxide.

10. The process of claim 7 wherein the reaction product after the additional step of converting at least a portion of the free fatty acids to fatty acid salts has an acid value less than 5 mg KOH/g.

11. The process of claim 1 wherein the epoxidized fatty acid alkyl esters have an oxirane oxygen content of from 2 to 12% by weight.

12. The process of claim 1 wherein the one or more epoxidized fatty acid triglycerides are obtained by epoxidizing one or more unsaturated fatty acid triglycerides with an organic peracid or a combination of an organic acid and hydrogen peroxide.

13. The process of claim 12, wherein the one or more epoxidized fatty acid triglycerides are obtained by epoxidizing one or more unsaturated fatty acid triglycerides with an organic peracid selected from the group consisting of peracetic acid and performic acid or a combination of an organic acid selected from the group consisting of acetic acid and formic acid and hydrogen peroxide.

14. The process of claim 12 wherein the epoxidation is catalyzed by at least one catalyst selected from the group consisting of acids, metal catalysts and enzymes.

15. The process of claim 1 wherein at least 1 mole monohydric alcohol per mole of fatty acid in the epoxidized fatty acid triglyceride is used.

16. The process of claim 1 wherein at least 1.2 moles monohydric alcohol per mole of fatty acid in the epoxidized fatty acid triglyceride is used.

17. The process of claim 1 wherein the one or more monohydric alcohols are added over a period of time to a mixture of the epoxidized fatty acid triglycerides and enzyme.

18. The process of claim 17, wherein the period of time is 8 hours or longer.

19. The process of claim 1 wherein less than 10 weight % water, based on the total weight of epoxidized fatty acid triglyceride(s) and alcohol, is present during the reacting.

20. The process of claim 1 wherein the epoxidized fatty acid triglyceride(s) is selected from the group consisting of epoxidized vegetable oils and fats, epoxidized animal oils and fats and combinations thereof.

21. The process of claim 1 wherein the epoxidized fatty acid triglyceride(s) is or are selected from the group consisting of epoxidized algae oil, epoxidized canola oil, epoxidized coconut oil, epoxidized castor oil, epoxidized corn oil, epoxidized cottonseed oil, epoxidized flax oil, epoxidized fish oil, epoxidized grapeseed oil, epoxidized hemp oil, epoxidized jatropha oil, epoxidized jojoba oil, epoxidized mustard oil, epoxidized canola oil, epoxidized palm oil, epoxidized palm stearin, epoxidized rapeseed oil, epoxidized safflower oil, epoxidized soybean oil, epoxidized sunflower oil, epoxidized tall oil, epoxidized olive oil, epoxidized tallow, epoxidized lard, epoxidized chicken fat, epoxidized linseed oil, epoxidized tung oil, epoxidized linseed oil, epoxidized tung oil and combinations thereof.

22. The process of claim 1 wherein the process is carried out in a batch mode, a semi-continuous mode or a continuous mode.

23. The process of claim 1 wherein the reacting is carried out under conditions effective to provide a reaction product comprised of 1% to 99% by weight epoxidized fatty acid triglycerides, based on the total weight of epoxidized fatty acid triglycerides, epoxidized fatty acid monoglycerides, epoxidized fatty acid diglycerides and epoxidized fatty acid alkyl esters present in the reaction product.

24. The process of claim 1 wherein the reacting is carried out under conditions effective to provide a reaction product comprised of 30% to 70% by weight epoxidized fatty acid triglycerides, based on the total weight of epoxidized fatty acid triglycerides, epoxidized fatty acid monoglycerides, epoxidized fatty acid diglycerides and epoxidized fatty acid alkyl esters present in the reaction product.

25. The process of claim 1 wherein the reacting is carried out in a two phase system wherein a first phase is comprised of the alcohol(s), enzyme, water and glycerol generated during transesterification and a second phase is comprised of the epoxidized fatty acid triglycerides and/or the epoxidized fatty acid alkyl esters.

26. The process of claim 25, wherein the first phase and the second phase are mixed during the reacting using high shear mixing.

27. The process of claim 25, wherein the process is conducted in a countercurrent mode.

28. The process of claim 25 wherein following the reacting the first phase is separated from the second phase and then re-used, in whole or in part, in a further transesterification reaction.

29. The process of claim 25 wherein glycerol is recovered from the first phase following the reacting.

30. The process of claim 25 wherein following the reacting the second phase is separated from the first phase and treated with an alkaline agent to reduce the level of free fatty acids present in the second phase.

31. The process of claim 25 wherein following the reacting the second phase is separated from the first phase and further treated with an immobilized lipase and alcohol to increase the content of epoxidized fatty acid alkyl esters.

32. A process of making epoxidized fatty acid alkyl esters, wherein the process comprises reacting a mixture of one or more unsaturated fatty acid triglycerides, one or more alcohols, and one or more active oxygen sources in the presence of at least one enzyme having transesterification activity and at least one enzyme having epoxidation activity or at least one enzyme having both transesterification activity and epoxidation activity.

33. The process of claim 32, wherein the active oxygen source(s) is or are selected from the group consisting of hydrogen peroxide and percarboxylic acids.

34. The process of claim 32 wherein the reacting is carried out under conditions effective to provide a reaction product comprised of 1% to 99% by weight epoxidized fatty acid triglycerides, based on the total weight of epoxidized fatty acid triglycerides, epoxidized fatty acid monoglycerides, epoxidized fatty acid diglycerides and epoxidized fatty acid alkyl esters present in the reaction product.

35. The process of claim 32 wherein the reacting is carried out under conditions effective to provide a reaction product comprised of 30% to 70% by weight epoxidized fatty acid triglycerides, based on the total weight of epoxidized fatty acid triglycerides, epoxidized fatty acid monoglycerides, epoxidized fatty acid diglycerides and epoxidized fatty acid alkyl esters present in the reaction product.

36. (canceled)

37. (canceled)

38. (canceled)
